SEQUENCE LISTING (5 sequences)

SEQ ID NO: 1 - protein, 309 aa, Escherichia coli [sequence omitted]

SEQ ID NO: 2 - protein, 23 aa, Escherichia coli
MNKKVLTLSAVMASMLFGAAAHA

SEQ ID NO: 3 - protein, 332 aa, Escherichia coli
MNKKVLTLSAVMASMLFGAAAHAADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGHPDAEARTTYVIKELNDKGIKTEQLQLDTAMWDTAQAKDKMDAWLSGPNANKIEVVIANNDAMAMGAVEALKAHNKSSIPVFGVDALPEALALVKSGALAGTVLNDANNQAKATFDLAKNLADGKGAADGTNWKIDNKVVRVPYVGVDKDNLAEFSKK

SEQ ID NO: 4 - protein, 317 aa, Artificial Sequence
  Preferred GBP molecule of the invention
MPKPQQFMADTRIGVTIYKYDDNFMSVVRKAIEQDAKAAPDVQLLMNDSQNDQSKQNDQIDVLLAKGVKALAINLVDPAAAGTVIEKARGQNVPVVFFNKEPSRKALDSYDKAYYVGTDSKESGIIQGDLIAKHWAANQGWDLNKDGQIQFVLLKGEPGCPDAEARTTYVIKELNDKGIKTEQLQLDTAMWDTAQAKDKMDAWLSGPNANKIEVVIANNDRMAMGAVEALKAHNKSSIPVFGVDASPEALALVKSGALAGTVLNDANNQAKATFDLAKNLADGKGAADGTNWKIDNKVVRVPYVGVDKDNLAEFSKK

SEQ ID NO: 5 - DNA, 954 bp, Artificial Sequence
  nucleic acid sequence of SEQ ID NO. 4
atgccaaaacctcagcagtttatggctgatactcgcattggtgtaacaatctataagtacgacgataactttatgtctgtagtgcgcaaggctattgagcaagatgcgaaagccgcgccagatgttcagctgctgatgaatgattctcagaatgaccagtccaagcagaacgatcagatcgacgtattgctggcgaaaggggtgaaggcactggcaatcaacctggttgacccggcagctgcgggtacggtgattgagaaagcgcgtgggcaaaacgtgccggtggttttcttcaacaaagaaccgtctcgtaaggcgctggatagctacgacaaagcctactacgttggcactgactccaaagagtccggcattattcaaggcgatttgattgctaaacactgggcggcgaatcagggttgggatctgaacaaagacggtcagattcagttcgtactgctgaaaggtgaaccgggctgtccggatgcagaagcacgtaccacttacgtgattaaggaattgaacgataaaggcatcaaaactgaacagttacagttagataccgcaatgtgggacaccgctcaggcgaaagataagatggacgcctggctgtctggcccgaacgccaacaaaatcgaagtggttatcgccaacaacgatcgcatggcaatgggcgcggttgaagcgctgaaagcacacaacaagtccagcattccggtgtttggcgtcgatgcgagcccagaagcgctggcgctggtgaaatccggtgcactggcgggcaccgtactgaacgatgctaacaaccaggcgaaagcgacctttgatctggcgaaaaacctggccgatggtaaaggtgcggctgatggcaccaactggaaaatcgacaacaaagtggtccgcgtaccttatgttggcgtagataaagacaacctggctgaattcagcaagaaataa

(19) United States
(12) Patent Application Publication
(10) Pub. No.: US 2012/0232251 A1
(43) Pub. Date: Sep. 13, 2012

(54) GLUCOSE SENSOR

(75) Inventors: John Pickup, London (GB); Faaizah Khan, London (GB)

(73) Assignee: King's College London, London (GB)

(21) Appl. No.: 13/509,565

(22) PCT Filed: Nov. 11, 2010
(86) PCT No.: PCT/GB2010/002076

§ 371 Date: May 11, 2012

(30) Foreign Application Priority Data
Nov. 13, 2009 (GB) 0920013.0

(51) Int. Cl. C07K 14/435; C07H 21/04

(52) U.S. Cl. 530/350; 536/23.5

(57) ABSTRACT

The invention relates to a glucose binding protein comprising amino acid mutations relative to the wild type sequence at the following positions: (i) H 152, (ii) A213; and (iii) L238 wherein the mutation at position H 152 is H152C. The invention further relates to such a glucose binding protein comprising the mutations H152C, A213R and L238S, in particular when linked to an environmentally sensitive dye such as badan.

### FIELD OF THE INVENTION

The invention relates to a glucose sensor based on the bacterial glucose/galactose binding protein (GBP).

### BACKGROUND TO THE INVENTION

Currently available sensors used in clinical practice for continuous glucose monitoring (CGM) in diabetes are subcutaneously implanted needle-type devices that are either amperometric enzyme electrodes, or microdialysis probes which sample interstitial fluid and deliver it to an ex vivo biosensor [1-3]. Both sensor types are based on immobilized glucose oxidase and the electrochemical detection of either hydrogen peroxide or electrons directly coupled to an underlying electrode via a molecular mediator [4]. Whilst evidence for the clinical utility of CGM is now accumulating [5], electrochemical glucose sensors suffer from impaired responses in vivo which necessitates frequent calibration, and contributes to sub-optimal accuracy [6, 7]. The likely reasons for poor CGM performance (in addition to time lags between blood and interstitial fluid) include electro-active interfering substances in vivo, coating of the implanted sensor by protein and cells (restricting glucose and oxygen access) and varying blood flow which changes tissue oxygen tension [6].

New technology for glucose sensing is therefore needed which is not based on electrochemistry and/or glucose oxidase. Fluorescence techniques for glucose sensing have the general advantages of sensitivity and independence from electro-active interference. Moreover, fluorescence lifetime can be measured as well as intensity [14,15] and this is not influenced by light scattering and by fluorophore concentration. Thus, such sensors promise more stable operation in vivo, as sensor coating or encapsulation in the tissues by protein or cells may diminish apparent fluorophore concentration and fluorescence intensity but not lifetime.

Amongst the potential receptors for glucose for use in optical sensors, fluorescence-labelled bacterial glucose/galactose-binding protein (GBP) has received considerable attention [16-26]. The protein structure of GBP has been provided by X-ray crystallography [27]. There is a marked conformational change in GBP which occurs on glucose binding, which can be monitored by fluorophore labelling of GBP and measurement of either fluorescence resonance energy transfer or the fluorescence changes of an environmentally sensitive dye attached near the binding site. In the latter option, changes in polarity and thus fluorescence occur as the lobes of GBP close round the dye on glucose binding [25,26].

A sensing strategy based on engineered GBP covalently linked to an environmentally sensitive dye, badan (6-bromoacetyl-2-dimethylaminonaphthalene), at position 152 near the binding site of glucose has been described where there was a large (300%) maximal increase in fluorescence intensity and 200% increase in lifetime [25,26] on addition of glucose. This GBP-badan system can be encapsulated within nano-engineered films formed by the layer-by-layer technique to create glucose micro-sensors, where glucose responses were monitored by fluorescence lifetime imaging microscopy (FLIM) [26]. The disadvantage of the system was that the binding constant for glucose (Kd) was in the micromolar range, making future clinical measurement impossible because of the common pathophysiological glycaemic range in diabetes of up to about 30 mM.

Certain known sensors are based on glucose/galactose binding protein (GGBP), which undergoes a marked conformational change on binding glucose. It is shown that this change can be monitored by labelling the GGBP with a fluorescent probe either using fluorescence resonance energy transfer or with an environmentally sensitive dye. When an environmentally sensitive dye is attached near the binding site, glucose binding causes polarity to decrease around the fluorophore as the lobes of the protein fold round the fluorophore, thus altering the fluorescence intensity in a glucose-dependent manner. Suitably badan is used as the environmentally sensitive dye, attached to a mutant of GGBP with histidine at position 152 changed to cysteine. We have shown that both intensity and lifetime increase by 300 and 200% respectively by addition of glucose (Khan et al, 2008; Saxl et al., 2009). However, labelled H152C-GGBP-badan cannot be used to form a CGM system for clinical applications because the binding constant is low at about 0.005 mM and saturation achieved at about 0.01 mM glucose.

Saxl et al (Biosensors and Bioelectronics 2009 1424 pages 3229 to 3324) disclose fluorescence lifetime spectroscopy and imaging of nano-engineered glucose sensor microcapsules based on glucose/glactose-binding protein. The protein studied in this publication is the H152C mutant GBP. This is a single point mutant of GBP. The only other mutation mentioned in this document is the F16A mutation. The GBP disclosed in this publication has an operating range which is relatively restricted and which is an order of magnitude below the typical patho-physiological range of blood glucose concentrations in diabetes. This is a serious drawback with the GBP disclosed.

Khan et al (BBRC 2008 Volume 365 pages 102 to 106) disclose a fluorescence-based sensing of glucose using engineered glucose/galactose-binding protein. This publication presents a comparison of fluorescence energy transfer and environmentally sensitive dye labelling strategies. In particular, the study discloses GBP with an M182C mutation linked to badan, and also discloses GBP with an H152C mutation linked to badan. None of the GBP moieties studied in their publication exhibited a Kd of greater than 2.35 micromolar. Molecules having a Kd for glucose of this level are not useful for clinical applications. This is a problem in the art.

Amiss et al (Protein Science 2007 Volume 16 pages 2350 to 2359) disclose engineering and rapid selection of a low affinity glucose/galactose binding protein for a glucose biosensor. This publication discloses a GBP having a single mutation of A213R. This molecule is disclosed to have a lowered glucose affinity of approximately 1 millimolar. Several other combination mutants are disclosed in this document, including an E149C A213R L238S triple mutant. Due to the desirable characteristics of this triple mutant, no further screening or selection studies were undertaken in this publication. However, this triple mutant exhibits only a fifty percent change in fluorescence intensity upon glucose binding when coupled to the IANBD fluorophore. Certain publications by Pitner et al have disclosed similar or identical glucose binding proteins having E149C, A213R and L238S mutations. However, these suffered from the same drawback of having only a fifty percent change of the fluorescence intensity upon glucose binding. This is a problem in the art.

Satins et al. mutated GGBP at three different position and attached various fluorophores at each position (M182C, H152C and G148C), Tolosa et al mutated the Gln at position 26 to Cys (Q26C) and Ge et al made the mutant L255C, all of which had binding constants in the micromolar range which is not suitable for physiological glucose monitoring. Hseih et al reported the E149C/A213S/L238S mutant; this has a Kd of 0.5 mM.

Thomas et al. reported two sensors based on E149C/A213C/L238S(GGBP) attached to Nile red (Thomas et al 2006) and E149C/A213R/L238S attached to benzothiazolium squarine derivatives (Thomas et al 2007) which can be used in the physiological glucose range having a Kd of 7 and 12 mM respectively. But in both cases, the actual fluorescence change is only 50%. Sakaguchi-Mukami et al reported mutations F16A and D14E/F16A, with the highest Kd being 3.9 mM for the latter protein but their method of assessment of glucose responsiveness is based on the autofluorescence of the protein, with a very small fluorescence change of about 10% making the system less sensitive, less robust and therefore unsuitable for clinical glucose sensing.

The present invention seeks to overcome problems associated with the prior art.

### SUMMARY OF THE INVENTION

Known glucose sensors have suffered from problems and drawbacks regarding their sensitivity and their response, together with their effective operating range. Typically sensors are developed towards ever greater sensitivity and therefore towards ever lower Kd values. Known sensors have also suffered from inadequate changes in fluorescence characteristics upon glucose binding.

The present inventors addressed these problems by studying GBP mutants and their characteristics and making novel combinations of mutations together with alternate dye strategies in order to create an improved glucose sensor.

The inventors arrived at a mutant of GBP covalently attached to a fluorophore such as badan which has an extended Kd and operating range suitable for clinical use. Both fluorescence intensity and lifetime change markedly on addition of glucose. The sensing system operates well in serum and is therefore a robust method for developing CGM in vivo. The invention is based on these striking findings.

Thus in one aspect the invention provides a glucose binding protein comprising amino acid mutations relative to the wild type sequence at the following positions:

### (i) H152,

### (ii) A213; and

(iii) L238
wherein the mutation at position H152 is H152C.

In another aspect, the invention relates to a glucose binding protein as described above comprising the mutations H152C, A213R and L238S.

In another aspect, the invention relates to a glucose binding protein as described above linked to an environmentally sensitive dye.

Suitably the environmentally sensitive dye comprises badan (6-bromoacetyl-2-dimethylaminonaphthalene).

In another aspect, the invention relates to a microcapsule comprising a glucose binding protein as described above.

In another aspect, the invention relates to a fibre optic strand comprising a glucose binding protein as described above, or a microcapsule as described above, attached thereto.

In another aspect, the invention relates to a nucleic acid encoding a glucose binding protein as described above.

In another aspect, the invention relates to a method of assessing glucose concentration in a system comprising monitoring the fluorescence of a glucose binding protein as described above in said system. Suitably said system comprises serum.

In another aspect, the invention relates to a method as described above wherein monitoring fluorescence comprises measuring fluorescence lifetime.

In another aspect, the invention relates to a method for Ni chelation of polystyrene beads, the method comprising:

a) providing polystyrene beads in a polar aprotic solvent, such as DMSO
b) contacting the beads with a reagent for activating carboxylic acids
c) incubating to permit activation to take place
d) removing solvent and activating reagent
e) suspending beads in alkaline carbonate buffer
f) contacting the beads with a metal chelator
g) incubating to allow chelation
h) contacting the beads with a source of Ni ions, such as Ni sulphite; and
i) contacting the beads with a quenching reagent, such as hydroxylamine.

In another aspect, the invention relates to a Ni chelated polystyrene bead.

In another aspect, the invention relates to a Ni chelated polystyrene bead produced by the method as described above.

In another aspect, the invention relates to a method for immobilising a GBP as described above, said method comprising contacting a Ni chelated polystyrene bead as described above with a GBP as described above and incubating to allow immobilisation.

In one aspect the invention provides a recombinant glucose/galactose binding protein (GBP) which is mutated relative to the wild type sequence at positions H152, A213 and L238.

Suitably position H152 is C.

Suitably position A213 is R.

Suitably position L238 is S.

In another aspect, the invention relates to use of a GBP as described above, for example in determination of glucose concentration and/or monitoring.

### DETAILED DESCRIPTION OF THE INVENTION

The preferred GBP of the invention possesses three mutations (H152, A213 and L238). This is the first time that such a triple mutant GBP has been described. Suitably the GBP of the invention is attached to an environmentally sensitive dye such as badan. Suitably this attachment is via H152 mutated to cysteine (H152C).

It should be noted that, despite considerable efforts and research investment in this high profile field, the present inventors are the first to describe this GBP mutant.

The science of protein engineering is often unpredictable. More specifically, engineering of GBP in order to manipulate its various different functions and properties is especially unpredictable. Computer based predictions of the behaviour of particular GBP mutants can be unreliable, and frequently incorrect. The inventors have arrived at the GBPs of the invention through a combination of insights and experiments, whose results would not have been predicted by unsophisticated computer simulations.

We synthesized mutants of glucose/galactose binding protein (GBP) labelled with the environmentally sensitive fluorophore badan, with an aim of producing a fluorescence-based glucose sensing system with an operating range compatible with continuous glucose monitoring in patients with diabetes mellitus. From five mutants tested, the triple mutant H152C/A213/L238S-badan showed a large (200%) maximal increase in fluorescence intensity on glucose addition, with a binding constant (K_{d}) of 11 mM, an operating range of approximately 1-100 mM and similar responses in buffer and serum. The fluorescence lifetime of this mutant also increased markedly on glucose addition. We conclude that GBP mutant H152C/A213/L238S, most suitably when labelled with badan, finds application as a robust sensor for glucose sensing, in particular in vivo glucose monitoring in diabetes.

The invention provides a fluorescence-based glucose sensing system using a mutant of GBP as the glucose receptor. Suitably the system of the invention has an operating range of about 1-100 mM. This has the advantage of being suitable for application in a biosensor used in the management of diabetes.

Suitably the system of the invention has a binding constant (K_{d}) of 11 mM.

Suitably the system of the invention has similar responses in buffer and serum.

The invention may be applied in vitro, for example to analyse in vitro samples.

The invention may be applied in vivo, for example by implantation (e.g. ‘smart tattoos’) or by topical introduction into or onto the subject being analysed.

A known H152C-badan mutant of GBP displays a large glucose-induced fluorescence intensity and lifetime change [9, 25,26] but with a K_{d} of approximately 2.5-5 μM. The site selected for attachment of badan, H152C, is located in the binding pocket of the protein and has been previously reported to show a fluorescence change with the environmentally sensitive dyes IANBD (N-(2-(iodoacetoxy)ethyl)-N-methyl)amino-7-nitrobenz-2-oxa-1,3-diazole) [16] or MDCC (N-[2-(1-maleimidyl)ethyl]-7-(diethylamino)coumarin-3-carbozamide) [18] but also with a K_{d} in the micromolar range.

Badan is an environmentally (polarity) sensitive fluorophore which when attached near the binding pocket of GBP shows a large change in its fluorescence intensity and lifetime when glucose binds [25], likely due to the closing of the two lobes of the protein around glucose, causing the environment of the fluorophore to become more hydrophobic. Knowledge of the tertiary structure of GBP [27] indicates some potential dye attachment sites at amino acid residues predicted to undergo large changes in environment upon ligand binding [29,30]. However, the fluorophore interaction with solvent and protein are complex and the magnitude of the fluorescence change with glucose for a particular dye and protein mutant cannot be readily predicted.

In order to alter the binding response of GBP and extend the operating range into the pathophysiological range, several combinations of mutation sites were examined in arriving at the present invention. It has been previously reported that the mutant L238S weakens the binding of glucose to GBP by about ten-fold [31]. Sakaguchi-Mukami et al [32] showed that the mutation F16A increases the K_{d} of GBP from 0.2 μM to 200 μM and the two-mutant D14E/F16A extended the K_{d} to 3.9 mM, when monitored by the autofluorescence of GBP.

Surprisingly, we found that the combination of the F16A mutation with H152C abolished the binding of glucose to GBP-badan completely, the protein showing no change of fluorescence intensity on glucose addition up to 1M. The triple mutation of H152C/A213R/L238S-badan, however, had a K_{d} of 11 mM in PBS.

Whilst in most previous reports of glucose sensors based on GBP, glucose monitoring could only be accomplished in the micromolar range, two reported systems based on GBP attached to Nile red [22] and benzothiazolium squaraine [23] derivatives had a Kₐ of 7 and 12 mM respectively. However, in both cases the fluorescence change was only about 50%, compared to the system described here (particularly when labelled with the preferred badan dye) which had a 200% change at maximal glucose levels.

We also show that the fluorescence responses of our system are similar in serum and buffer, and thus the combination of operation in biological fluid and the very high signal change demonstrates that our system is a more robust technology, particularly for in vivo monitoring, than known sensors.

Fluorescence lifetime measurement is a technology that is particularly suitable for in vivo monitoring in diabetes management and for the spatial resolution of sensing with FLIM because of the independence of lifetime from the concentration of the dye, and the relatively small effect of photobleaching and scatter. In this respect, it is significant that we show not only a large change in fluorescence intensity but also lifetime on addition of glucose. As with H152C-badan, a model with two lifetimes best fitted the decay curves, and we again found that glucose addition caused an increase in the proportion of the long lifetime component and a decrease of a short lifetime component, which we have previously discussed is probably a reflection of increase in the closed, glucose-bound form and decrease in the open, glucose-unbound form of GBP [26].

Commonly observed blood glucose levels in diabetes are from about 1 (in the hypoglycaemic range) to about 30 mM (in the hyperglycaemic range). Thus suitably the sensor of the invention is responsive across this range of concentrations.

Mutants of GGBP with altered binding properties as described herein increase the Kd of GGBP and allow the protein to be used for the construction of glucose sensing devices/systems.

We describe synthesis of a mutant protein based on engineered glucose/galactose binding protein (GBP) that has a binding constant (Kd) of approximately 11 mM and is thus suitable for measurement of glucose in the pathophysiological range, for example for use in the monitoring of glucose in human diabetes mellitus.

The preferred mutant [H152C/A213/L238S(GGBP)] is described, which has the amino acid histidine at position 152 mutated to the amino acid cysteine, alanine at position 213 mutated to arginine and lysine at position 238 mutated to serine.

### DEFINITIONS

The term ‘comprises’ (comprise, comprising) should be understood to have its normal meaning in the art, i.e. that the stated feature or group of features is included, but that the term does not exclude any other stated feature or group of features from also being present.

Abbreviations used include CGM, continuous glucose monitoring; GBP, glucose/galactose-binding protein; IANBD, N-(2-(iodoacetoxy)ethyl)-N-methyl)amino-7-nitrobenz-2-oxa-1,3-diazole; MDCC, N-[2-(1-maleimidyl)ethyl]-7-(diethylamino)coumarin-3-carbozamide)

### Dyes

Suitably the mutated GBP of the invention is linked to an environmentally sensitive dye such as an environmentally sensitive fluorophore. Such an entity suitably displays a change in fluorescence intensity in a hydrophobic environment, such as the interior of a protein, compared to a hydrophilic environment, such as the exterior of a protein. Plus, when the GBP molecule binds glucose, it undergoes a distinct and marked conformational change. This conformational change results in the environmentally sensitive dye being subjected to a change in its chemical environment, and therefore changing its fluorescent behaviour. In this manner, the binding of glucose to the GBP of the invention can be read out by monitoring the fluorescent behaviour of the environmentally sensitive dye, and thereby give a direct and reliable indication of glucose levels in the system being studied.

Numerous environmentally sensitive dyes, such as polarity sensitive dyes, may be useful in the invention. For example, members of the family of dimethylaminonaphthalene dyes such as acrylodan, prodan or laurdan would be expected to show a similar change in fluorescence, but the precise extent of change in fluorescence with these dyes would need to be checked by the skilled operator for optimal performance. Other environment sensitive dyes (not of the badan family) such as Nile red, Nile blue and benzo squaraine derivatives may also find application in the invention when attached to GBP, but of course their response at position 152 in combination with the other two mutants (A213R, L238S) of the preferred GBP of the invention would advantageously be checked by the skilled operator.

Most suitably the environmentally sensitive dye comprises a diamino-naphthalene moiety. Suitably the environmentally sensitive dye is badan (6-bromoacetyl-2-dimethylaminonaphthalene).

Suitably the environmentally sensitive dye is attached to the glucose binding protein at amino acid 152. Suitably this attachment is via a cysteine residue substituted for the wild type histidine residue at amino acid position 152 (H152C).

### Tags

In some embodiments, it may be advantageous to tag GBP of the invention. This may be useful in facilitating immobilisation. This may be useful in easing purification. Suitably, the tag used may be a histidine tag such as a 6H is tag.

### Microcapsules

The GBP of the invention may suitably be formulated into microcapsules. This has the advantage of making the GBP of the invention readily available for use in a variety of different applications. Suitably such microcapsules are made by the “layer by layer” technique known in the art. In case any guidance is needed, reference is made to Zhi, Z-L and Haynie, D. T. Straightforward and Effective Protein Encapsulation in Polypeptide-based Artificial Cells (2006), Artificial Cells, Blood Substitutes, and Biotechnology, 34: 189-203 for the express teaching of the “layer-by-layer” technique. Moreover, a method for encapsulation of badan-GBP in layer-by-layer films is described in: Saxl, T., Khan, F., Matthews, D. R., Zhi, Z-L., Rolinski, O., Ameer-Beg, S., Pickup, J. C. Fluorescence lifetime spectroscopy and imaging of nano-engineered glucose sensor microcapsules based on glucose/galactose-binding protein. Biosens. Bioelectron. 24 (2009) 3229-34, which is referred to expressly for the description of this encapsulation technique.

Microcapsules of the invention may be implantable into a human or animal subject.

Microcapsules according to the invention may be attachable to an optical fibre, either directly or indirectly, in order to form a biosensor according to the invention. Typically the end of the optical fibre would be provided with a gel or other such substrate, in which microcapsules comprising a GBP of the invention are suspended or embedded, thereby creating a fibre optic sensor according to the present invention.

The invention finds application in any environment in which it is required to monitor the presence or absence (and in particular the concentration) of glucose. The invention finds particular application in diabetes.

### REFERENCE SEQUENCE

When particular amino acid residues of glucose binding protein (GBP) are referred to using numeric addresses, the numbering is taken with reference to the wild type E. coli amino acid sequence (or to the nucleic acid sequence encoding same). In particular, for the avoidance of doubt, the wild type amino acid sequence of GBP is presented below:

ADTRIGVTIYKYDDNFMSVVRKAIEQDAKAAPDVQLLMNDSQNDQSKQNDQIDVLLAKGVKALAI
NLVDPAAAGTVIEKARGQNVPVVFFNKEPSRKALDSYDKAYYVGTDSKESGIIQGDLIAKHWAANQ
GWDLNKDGQIQFVLLKGEPGHPDAEARTTYVIKELNDKGIKTEQLQLDTAMWDTAQAKDKMDAW
LSGPNANKIEVVIANNDAMAMGAVEALKAHNKSSIPVFGVDALPEALALVKSGALAGTVLNDANN
QAKATFDLAKNLADGKGAADGTNWKIDNKVVRVPYVGVDKDNLAEFSKK

This is to be used as is well understood in the art to locate the residue of interest. This is not always a strict counting exercise—attention must be paid to the context. For example, if the protein of interest is of a slightly different length, then location of the correct residue in that sequence corresponding to (for example) H152 may require the sequences to be aligned and the equivalent or corresponding residue picked, rather than simply taking the 152nd residue of the sequence of interest. This is well within the ambit of the skilled reader.

It must be noted that the wild type E. coli GBP gene comprises a leader sequence. Typically, this leader sequence is discarded. In other words, suitably the recombinant GBP of the invention is synthesised without the leader sequence. For the avoidance of doubt, the leader sequence has the following amino acid composition: MNKKVLTLSAVMASMLFGAAAHA so that the corresponding GBP sequence including the leader sequence is:

MNKKVLTLSAVMASMLFGAAAHAADTRIGVTIYKYDDNFMSVVRKAIEQDAKAAPDVQLLMNDSQ
NDQSKQNDQIDVLLAKGVKALAINLVDPAAAGTVIEKARGQNVPVVFFNKEPSRKALDSYDKAYYV
GTDSKESGIIQGDLIAKHWAANQGWDLNKDGQIQFVLLKGEPGHPDAEARTTYVIKELNDKGIKTEQ
LQLDTAMWDTAQAKDKMDAWLSGPNANKIEVVIANNDAMAMGAVEALKAHNKSSIPVFGVDAL
PEALALVKSGALAGTVLNDANNQAKATFDLAKNLADGKGAADGTNWKIDNKVVRVPYVGVDKD
NLAEFSKK

Suitably references to the sequence of GBP herein relate to the GBP sequence without the leader sequence unless otherwise clear from the context.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, as a default alanine (A) may be used. Suitably the mutations used at particular site(s) are as set out herein.

A fragment is suitably at least 10 amino acids in length, suitably at least 25 amino acids, suitably at least 50 amino acids, suitably at least 100 amino acids, or suitably the majority of the GBP polypeptide of interest i.e. 155 amino acids or more, suitably at least 200 amino acids, suitably at least 250 amino acids, suitably at least 300 amino acids, suitably the entire 309 amino acids of the GBP sequence (i.e. excluding the leader sequence).

### Sequence Variation

The sensor of the invention may comprise sequence changes relative to the wild type sequence in addition to the key mutations described in more detail herein. Specifically the sensor of the invention may comprise sequence changes at sites which do not significantly compromise the function or operation of the sensor as described herein.

Sensor function may be easily tested by operating the sensor as described, such as in the examples section, in order to verify that function has not been abrogated or significantly altered.

Thus, provided that the sensor retains its function which can be easily tested as set out herein, sequence variations may be made in the sensor molecule relative to the wild type reference sequence.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In considering what mutations, substitutions or other such changes might be made relative to the wild type sequence, retention of the function of the sensor is paramount. Typically conservative amino acid substitutions would be less likely to adversely affect the function. Suitably the sensor of the invention varies from the wild type sequence only by conservative amino acid substitutions.

More specifically suitably the sensor of the invention should not be mutated at amino acid residues corresponding to the glucose binding pocket of the sensor molecule (other than those taught herein). The glucose binding pocket comprises amino acid residues which are distributed around the molecule and does not necessarily consist of a contiguous or linear amino acid sequence. In order to provide further guidance on this point, suitably the amino acid positions in the following table are considered to comprise the glucose binding pocket of the sensor molecule:

TABLE A

Amino acid residues in the binding pocket of GBP:
Tyr10, Asp 14, Phe16, Val19, Asn 91, Gly 148, Glu149, His 152,
Asp 154, Arg 158, Trp183, Asn 211, Ala213, Asp236, Asn 256,
Leu 238
Reference - Vyas, N. K., Vyas, M. N. and Quiocho, F. A.
The 3 Å resolution structure of a D-galactose-binding protein for
transport and chemotaxis in Escherichia coli (1983),
Proc. Nat. Acad. Sci. USA 80: 1792-1796.

Suitably the sensor molecule of the invention is not mutated at the residues of table A except as described for specific residues discussed herein. Suitably the sensor molecule of the invention has polypeptide sequence corresponding to the wild type GBP sequence at the residues of table A except as described. Suitably the sensor molecule of the invention has polypeptide sequence corresponding to the wild type GBP sequence at each of the residues of table A except as described.

Suitably the sensor molecule of the invention is not mutated at the residues neighbouring the residues of table A. By ‘neighbouring’ is meant immediately adjacent to or linked by peptide bond to. Thus each amino acid has two neighbours (other than the extreme N- or C-terminal amino acids which each have only one neighbour). This has the advantage of avoiding disruption to the immediate local environment of the residues known to be part of the glucose binding site.

A most preferred GBP molecule of the invention comprises, or consists of, the following sequence:

MPKPQQFMADTRIGVTIYKYDDNFMSVVRKAIEQDAKAAPDVQLLMNDSQNDQSKQNDQIDVLL
AKGVKALAINLVDPAAAGTVIEKARGQNVPVVFFNKEPSRKALDSYDKAYYVGTDSKESGIIQGDLI
AKHWAANQGWDLNKDGQIQFVLLKGEPGCPDAEARTTYVIKELNDKGIKTEQLQLDTAMWDTAQ
AKDKMDAWLSGPNANKIEVVIANNDRMAMGAVEALKAHNKSSIPVFGVDASPEALALVKSGALA
GTVLNDANNQAKATFDLAKNLADGKGAADGTNWKIDNKVVRVPYVGVDKDNLAEFSKK
(mutations underlined and the transglutaminase tag at N-terminal in bold)

The transglutaminase tag may be removed to leave the preferred GBP mutant sequence of the invention, or may be replaced with a different tag chosen by the operator.

A most preferred nucleic acid of the invention comprises, or consists of, the following sequence:

ATGCCAAAACCTCAGCAGTTTATGGCTGATACTCGCATTGGTGTAACAATCTATAAGTACGACGATAACT
TTATGTCTGTAGTGCGCAAGGCTATTGAGCAAGATGCGAAAGCCGCGCCAGATGTTCAGCTGCTGATGAA
TGATTCTCAGAATGACCAGTCCAAGCAGAACGATCAGATCGACGTATTGCTGGCGAAAGGGGTGAAGGCA
CTGGCAATCAACCTGGTTGACCCGGCAGCTGCGGGTACGGTGATTGAGAAAGCGCGTGGGCAAAACGTGC
CGGTGGTTTTCTTCAACAAAGAACCGTCTCGTAAGGCGCTGGATAGCTACGACAAAGCCTACTACGTTGG
CACTGACTCCAAAGAGTCCGGCATTATTCAAGGCGATTTGATTGCTAAACACTGGGCGGCGAATCAGGGT
TGGGATCTGAACAAAGACGGTCAGATTCAGTTCGTACTGCTGAAAGGTGAACCGGGCTGTCCGGATGCAG
AAGCACGTACCACTTACGTGATTAAGGAATTGAACGATAAAGGCATCAAAACTGAACAGTTACAGTTAGA
TACCGCAATGTGGGACACCGCTCAGGCGAAAGATAAGATGGACGCCTGGCTGTCTGGCCCGAACGCCAAC
AAAATCGAAGTGGTTATCGCCAACAACGATCGCATGGCAATGGGCGCGGTTGAAGCGCTGAAAGCACACA
ACAAGTCCAGCATTCCGGTGTTTGGCGTCGATGCGAGCCCAGAAGCGCTGGCGCTGGTGAAATCCGGTGC
ACTGGCGGGCACCGTACTGAACGATGCTAACAACCAGGCGAAAGCGACCTTTGATCTGGCGAAAAACCTG
GCCGATGGTAAAGGTGCGGCTGATGGCACCAACTGGAAAATCGACAACAAAGTGGTCCGCGTACCTTATG
TTGGCGTAGATAAAGACAACCTGGCTGAATTCAGCAAGAAATAA
(mutations underlined and the transglutaminase tag at N-term in bold)

### Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Mine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

In the context of the present document, a homologous amino acid sequence is taken to include an amino acid sequence which is at least 15, 20, 25, 30, 40, 50, 60, 70, 80 or 90% identical, preferably at least 95 or 98% identical at the amino acid level. Suitably this identity is assessed over at least 50 or 100, preferably 200, 300, or even more amino acids with the relevant polypeptide sequence(s) disclosed herein, most suitably with the full length progenitor (parent/wild type) GBP sequence.

Suitably, homology should be considered with respect to one or more of those regions of the sequence known to be essential for protein function rather than non-essential neighbouring sequences. This is especially important when considering homologous sequences from distantly related organisms.

Most suitably sequence identity should be judged across at least the glucose binding site of the amino acid sequence of E. coli GBP, or the corresponding region in an alternate GBP.

The same considerations apply to nucleic acid nucleotide sequences.

### Polynucleotides of the Invention

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

### Protein Expression and Purification

Proteins of the invention are typically made by recombinant means, for example as described below and in the examples. However they may also be made by synthetic means using techniques well known to skilled persons such as solid phase synthesis. Proteins of the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Clearly the fusion protein selected must not hinder the function of the GBP of the invention.

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### Immobilisation/Solid Phase Sensors

GBP-badan may be immobilized to a surface. This provides the advantage of permitting assay using a solid phase sensor.

Immobilisation may be via the interaction of a oligohistidine tag included on GBP to Ni chelated at a solid matrix.

The solid matrix may be any Ni-chelated substrate, for example Ni-chelated polystyrene beads or Ni-NTA (nitrilotriacetic acid) agarose resin/beads. NTA agarose beads can be purchased commercially. GBP according to the present invention such as GBP-badan may be immobilized in batches, for example to avoid storage of the conjugated polypeptide-dye GBP moiety.

The Ni chelation of polystyrene beads is disclosed herein. Thus in one embodiment the invention relates to a method for chelation of polystyrene beads such as Ni chelation of polystyrene beads, the method comprising:

a) providing polystyrene beads in a polar aprotic solvent, such as DMSO
b) contacting the beads with a reagent for activating carboxylic acids
c) incubating to permit activation to take place
d) removing solvent and activating reagent
e) suspending beads in alkaline carbonate buffer
f) contacting the beads with a metal chelator
g) incubating to allow chelation
h) contacting the beads with a source of Ni ions, such as Ni sulphite; and
i) contacting the beads with a quenching reagent, such as hydroxylamine.

The beads may be finally prepared for use by optionally removing reagents and suspending beads in buffer, such as phosphate buffered saline (PBS).

Providing polystyrene beads in a polar aprotic solvent, such as DMSO may comprise resuspending 5 mg polystyrene beads (AM-COOH, 100-200 mesh) in DMSO.

A reagent for activating carboxylic acids may comprise N-hydroxysuccinimide and EDC (ethyl dimethylaminopropyl carbodiimide).

Incubating to permit activation to take place may comprise stirring at RT (e.g. 18-22 Celsius) for 3 h.

Removing solvent and activating reagent may comprise centrifugation to remove the DMSO/activating reagent and may optionally further comprise washing beads with a phosphate buffer, such as twice.

Suspending beads in alkaline carbonate buffer may comprise resuspending in 1 ml 0.1M carbonate buffer pH 8.0.

Contacting the beads with a metal chelator may comprise adding bis(carboxymethyl)lysine metal chelator (such as to a final concentration of 15 mM).

Incubating to allow chelation may comprise stirring at RT (e.g. 18 to 22 Celsius) for 24 h.

Contacting the beads with a source of Ni ions, may comprise adding Ni sulphite (such as to a final concentration 150 mM).

Contacting the beads with a quenching reagent, may comprise 10 mM hydroxylamine for 1 hour (such as by adding 10 ul of 1M stock, 69 mg/ml).

Optionally removing reagents and suspending beads in buffer, may comprise centrifuging and wash twice in phosphate buffered saline (PBS).

To immobilise a GBP of the invention to the beads, they may be contacted with GBP-badan (e.g. at 50 uM in PBS) wherein said GBP comprises 6his tag, and incubated to permit immobilisation, for example by incubating at 4° C. overnight (e.g. approx. 12-16 hours).

In a most preferred embodiment the method may comprise

1. Resuspend 5 mg polystyrene beads (AM-COOH, 100-200 mesh) in DMSO
2. Add 1.5 mg N-hydroxysuccinimide and 3 mg EDC (ethyl dimethylaminopropyl carbodiimide). Stir at RT for 3 h
3. Centrifuge to remove the DMSO.
4. Wash beads with a phosphate buffer twice, and resuspend in 1 ml 0.1M carbonate buffer pH 8.0
5. Add 3.9 mg 15 mM bis(carboxymethyl)lysine metal chelator (final concentration 15 mM)

### 6. Stir at RT for 24 h

7. Add 3.9 mg Ni sulphite (final concentration 150 mM)
8. Quench with 10 mM hydroxylamine 1 hour (add 10 ul of 1M stock, 69 mg/ml) and optionally
9. Centrifuge and wash twice in PBS.
The beads may then be optionally loaded with GBP according to the present invention for example by

### 10. Add 200 ul GBP-badan (50 uM in PBS)

### 11. Incubate o/n 4° C.

### ADVANTAGES

The technical advantages offered by the GBPs of the present invention are many and varied. In particular, the GBPs of the invention offer excellent fluorescence intensity change upon glucose binding. Moreover, the GBPs of the invention have a binding constant (K_{d}) which is within the physiologically significant glucose concentration range of approximately one millimolar to approximately thirty millimolar. Thus, the GBPs of the invention combine for the first time the advantageous properties of excellent responsiveness to glucose binding (i.e. excellent change in fluorescent intensity on glucose binding) together with a dynamic range which maps well within the physiologically relevant range of glucose concentrations. First, it is an advantage of the invention that for the first time a useful molecular biosensor is provided which enables accurate and reliable readout of glucose concentrations, and exhibits glucose binding properties which enable robust and reliable responses to glucose concentrations at the hypoglycaemic normal and hyperglycaemic levels which are typically encountered in corresponding mammals such as humans.

Although there may be a reported mutation of GBP that has a similar Kd to the GBPs of the invention (Thomas), the maximal glucose-induced increase in fluorescence with the reported fluorophores in this work (a derivative of Nile Red or a squarine dye) is small (50%) compared to the mutant of GBP linked to badan which we report (200%). This combination is a major advantage for a glucose sensing system.

Although the A213 & L238 residues may have been shown to alter Kd, the result of this combination with H152C attached with Badan could not be predicted because although knowledge of the protein tertiary structure might allows one to select candidate attachment sites near the location predicted to undergo maximum change in environment on ligand binding, the dye used and its interaction with the solvent and the protein is complex and cannot be predicted.

Numerous workers have researched the problem of mutating GBP to extend the Kd and only one (Thomas 2007) approaches the Kd of GBPs of the present invention, and none have the large fluorescence increase with addition of glucose that we demonstrate. This illustrates that the tasks of choosing the mutation strategy and finding a suitable mutant protein with retained response are extremely challenging.

The mutants may be manufactured by any standard site-directed mutagenesis protocol.

H152C/A213/L238S(GGBP) can be labelled with an environmentally sensitive fluorophore dye such as badan by covalent linkage to the cysteine residue at position 152 and the large change in fluorescence is maintained even in biological fluid such as serum, making it ideal for clinical blood glucose monitoring.

Although certain mutants might be thought candidates to affect the Kd from a knowledge of the tertiary structure of GBP, the extent and the ability to retain a fluorescence response with glucose and the magnitude of this could not be predicted.

In addition, the results presented herein in support of the invention were better than expected in the sense the Kd was within the physiological range and the fluorescence intensity change with glucose was still very large (200% increase). This is further indication that performance of mutants cannot be anticipated from the prior art.

The badan labelled proteins/systems disclosed herein have a 200% or even 300% fluorescence intensity change making them a more sensitive and therefore robust system for glucose monitoring such as in vivo monitoring.

H152C/A213/L238S(GGBP) can be labelled with an environmentally sensitive fluorophore dye such as badan by covalent linkage to the cysteine residue at position 152 and that addition of glucose changes the fluorescence intensity of the GGBP by a large amount such as to provide a measurement system for glucose, e.g. for use as a monitor in diabetes mellitus.

H152C/A213/L238S(GGBP) can be labelled with an environmentally sensitive fluorophore dye such as badan by covalent linkage to the cysteine residue at position 152 and that addition of glucose changes the fluorescent lifetime of the GGBP by a large amount such as to provide a measurement system for glucose, e.g. for use as a monitor in diabetes mellitus.

### Further Applications

The invention relates to a fluorescence intensity- and lifetime-based glucose sensing system using an engineered mutant of glucose/galactose-binding protein with high k_{d}.

The invention finds application in any context in which glucose sensing is helpful or desired. In particular the invention finds application in aiding the diagnosis and/or monitoring of conditions such as diabetes mellitus.

Amongst the applications of GBP-badan for CGM is immobilization at the tip of a fibre-optic probe which can be implanted in the subcutaneous tissue and linked to an external recorder of fluorescence lifetime.

The invention also provides a CGM system based on fluorescence lifetime changes of H152C/A213/L238S(GGBP) that is especially suitable for use in diabetes. This has particular advantages because any coating of the sensor in vivo or diminution of GGBP-fluorophore intensity will not affect the fluorescence lifetime and its dependence of glucose concentrations, thus making such a CGM system more stable and accurate than electrochemically-based CGM systems.

H152C/A213/L238S(GGBP) labelled with an environmentally sensitive fluorophore such as badan can be incorporated into a fibre optic probe for the continuous monitoring of glucose in diabetes mellitus. Such a fibre optic based glucose sensor using H152C/A213/L238S(GGBP)-badan or another fluorophore attached to H152C/A213/L238S(GGBP) might monitor glucose concentrations in the interstitial fluid in the subcutaneous tissue or in the blood stream of subjects with diabetes.

### BRIEF DESCRIPTION OF THE FIGURES

FIG. 1 shows graphs of fluorescence emission spectra of GBP mutant H152C/A213R/L238S-badan in 0 and 150 mM glucose

FIG. 2 shows graphs of increase in fluorescence intensity of GBP mutants H152C-badan and H152C/A213R/L238S-badan with addition of glucose.

FIG. 3 shows graphs of Increase in fluorescence intensity of GBP mutant H152C/A213R/L238S-badan with the addition of glucose in PBS and serum.

FIG. 4 shows graphs of (a), biexponential fits to fluorescence decay curves of mutant H152C/A213R/L238-badan in zero and saturating glucose concentrations. (b), change in mean fluorescence lifetime of H152C/A213R/L238-badan with addition of glucose. Inset, the fractional contribution of lifetime states 3.1 and 0.9 ns plotted with increasing glucose concentrations.

The invention is now described by way of example. These examples are intended to be illustrative, and are not intended to limit the appended claims.

### EXAMPLES

### Overview

We use site-directed mutagenesis to create a number of mutants of GBP with a potentially higher K_{d} than known GBP mutants. These mutants were designed by the inventors.

Fluorescence intensity and lifetime measurements were then carried out on dye-labelled GBP mutants (such as badan-labelled GBP mutants) to select a glucose-responsive sensing molecule or system. The labelled GBP molecules disclosed herein are advantageously suitable for clinical use.

### Materials and Methods

Most chemicals used were purchased from Sigma-Aldrich (St Louis, USA). The pTZ18U-mgIB vector containing the GBP gene was a kind gift from Dr S D'Auria. The plasmid pET303/CT-His vector was purchased from Invitrogen (Paisley, UK): E. coli DH5α cells were used as host cells for plasmid proliferation. LB media supplemented by antibiotics (50 μg/ml of kanamycin or 100 μg/ml of ampicillin) were employed to grow cells. E. coli BL21(DE3) was from BD Biosciences (Franklin Lakes, N.J., USA). All restriction enzymes were purchased from New England Biolabs (Hitchen, UK). Quick-change site-directed mutagenesis kit was purchased from Stratagene (La Jolla, Calif., USA). The Rapid DNA ligation kit and long-template polymerase chain reaction (PCR) enzyme (Expand) were from Roche Applied Science (Basel, Switzerland). The kit used for plasmid extraction and Ni-NTA agarose was from Qiagen (West Sussex, UK) and the kit used to purify PCR products or restriction reactions was from Qbiogene (Morgan Irvine, Calif., USA). The fluorescent probe badan was from Invitrogen.

### Example 1

### Construction of Expression Vector pET303-GBP and Purification of Mutants of GBP

Details of the methodology are given in Khan of al. [25]. In brief, the GBP gene (mgIB) was isolated from the plasmid pTZ18U-mgIB by PCR and ligated into pET303/CT-His vector using a Rapid DNA ligation kit to form pET303-GBP. For the H152C, H152C/A213R, H152C/L238S and H152C/A213R/L238S mutants, pET303-GBP was used as a template. Site-directed mutagenesis was performed using the Quick-change mutagenesis kit with respective primers for each mutation. DNA sequencing data verified the presence of the desired mutations. A single colony of E. coli BL21(DE3) transformed with the pET303-GBP plasmid containing various mutants was inoculated in LB media containing 100 μg/ml of ampicillin and grown at 37° C. Expression of the proteins was induced by adding isopropyl-2-D-thiogalactopyranoside to a final concentration of 1 mM. Bacterial cells were lysed and the cell extract was clarified by centrifugation. Affinity chromatography was performed in a glass column packed with 5 ml Ni-NTA agarose. The protein was eluted with buffer containing 250 mM imidazole. The purity of GBP was determined by SDS-PAGE using 10% acrylamide gels that were viewed by Coomassie Blue staining.

### Example 2

### Fluorophore Labelling

To label GBP mutants with badan, 50 μM protein was dissolved in 5 mM Tris(2-carboxyethyl)phosphine in phosphate-buffered saline (PBS) pH 7.4, and then a 10-fold excess of dye (500 μM) was added and the mixture incubated overnight at 4° C., after which it was purified on a Sephadex G-25 gel-filtration column.

### Example 3

### Serum Preparation

Venous blood samples from healthy volunteers were collected in Vacuette tubes. The specimens were incubated at room temperature for 4 days to allow blood clotting and glycolysis. The samples were then centrifuged at 3500 g for 20 min and serum removed, pooled and stored until use. The glucose concentrations of serum samples were determined using a hexokinase-based assay (Sigma).

### Example 4

### Steady-State Fluorescence Measurements

Steady-state fluorescence intensity was recorded on a Perkin-Elmer LS50B fluorimeter (Perkin Elmer Instruments, Beaconsfield, UK). The excitation and emission wavelengths of badan were 400 and 550 nm respectively. All data were obtained at room temperature using quartz cuvettes with sample volume of 100 μL. The labelled protein was incubated with increasing amounts of D-glucose for 15-20 min at room temperature before fluorescence was recorded.

### Example 5

### Fluorescence Lifetime Measurements

All lifetime experiments were performed in PBS. Glucose was added sequentially to a cuvette containing a 100 μl solution of 5 μM GBP-badan. Excitation was provided by a bandpass filtered (417±10 nm), supercontinuum (white-light) laser (Fianium, UK). Emission was bandpass filtered (542±50 nm), detected using a fast photomultiplier tube (PMH100 Becker and Hickl GmbH, Berlin, Germany) and processed by time-correlated single-photon counting (TCSPC) electronics (SPC830, Becker and Hickl GmbH, Berlin, Germany).

### Example 6

### Data Analysis

The binding constant, K_{d}, was calculated from the sigmoidal dose-response curves using Prism 5 software (GraphPad, San Diego, Calif., USA). Lifetime values were obtained from fluorescence transients using the TR12 analysis package (courtesy of Dr. Paul Barber, Gray Cancer Institute of Radiation Oncology and Biology, Oxford University, Oxford, UK). Global analysis was applied to the entire data set of twelve transients [28] as described for the H152C mutant previously [26].

### Example 7

### Fluorescence Intensity of GBP Mutants

In order to increase the K_{d} and thereby the operating range of a glucose-sensing system based on GBP, we engineered five candidate mutants of GBP-H152C, F16A/H152C, H152C/A213R, H152C/L238S and H152C/A213R/L238S—and labelled each of them at position 152C near the glucose binding site with the environmentally sensitive fluorophore, badan (covalently linked to the unique cysteine at this site via the maleimide derivative of the dye). The maximal percentage changes in fluorescence with the addition of glucose and the calculated K_{d} for each mutant are shown in Table 1, compared to the previously reported single mutant H152C-badan.

Mutant F16A/H152C-badan showed no change in fluorescence with increasing glucose concentration. The largest maximal increase in fluorescence intensity (˜500%) on glucose addition occurred with mutant H152C/A213R-badan. Mutants H152C/L238S-badan and H152C/A213R/L238S-badan were associated with fluorescent intensity enhancements of 200% at saturating glucose levels.

The K_{d} of mutants H152C/A213R-badan and H152C/L238S-badan was increased by a small amount compared to H152C-badan, from 0.005 mM to 0.6 mM (in both cases), but we found that the K_{d} of the triple mutant H152C/A213R/L238S-badan was markedly increased to 11 mM (Table 1).

TABLE 1
Fluorescence responses (maximal change in fluorescence intensity
with addition of glucose) and binding constants of mutants of GBP
lablled with badan

| GBP mutant | Δ Fluorescence (%) | K_{d} (mM) |
|---|---|---|
| H152C | 400 | 0.005 |
| H152C/F16A | 0 | — |
| H152C/A213R | 500 | 0.6 |
| H152C/L238S | 200 | 0.6 |
| H152C/A213R/L238S (in | 200 | 11 |
| PBS) | | |
| H152C/A213R/L238S | 180 | 14 |
| (in serum) | | |

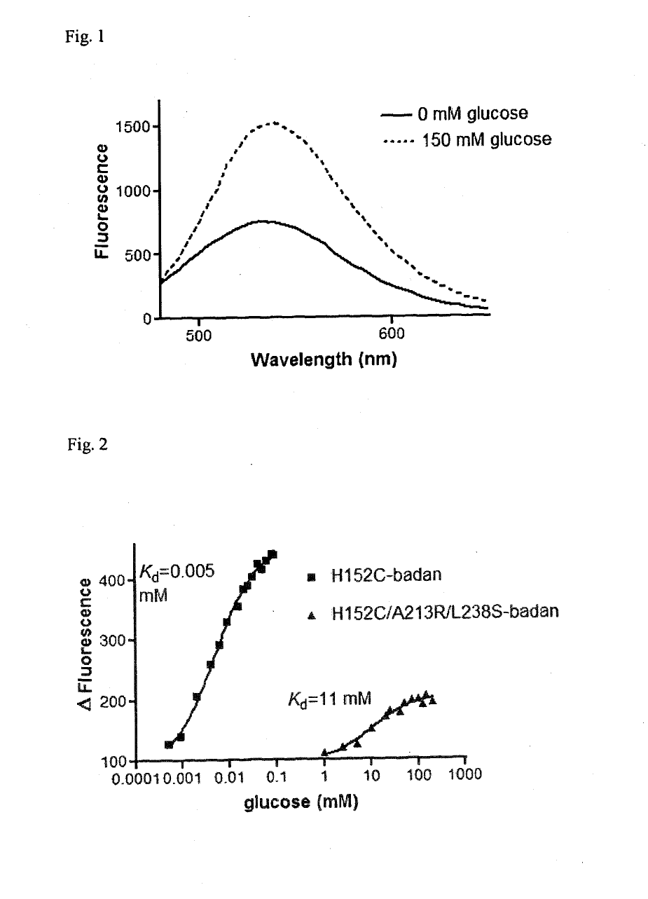

FIG. 1 shows the emission spectra of the mutant H152C/A213R/L238S-badan in the presence of 0 and 150 mM glucose, where an excitation maximum at 550 nm was maintained with the increased fluorescence intensity at high glucose level.

FIG. 2 compares the glucose response curve of the single mutant H152C-badan with the triple mutant H152C/A213R/L238S-badan, and shows that the three mutations are associated with an operating range of about 1-100 mM glucose concentration and a three-order of magnitude change in K_{d}. Thus, this GBP-badan conjugate has the potential to detect glucose in the pathophysiological range and would be suitable for eventual in vivo applications.

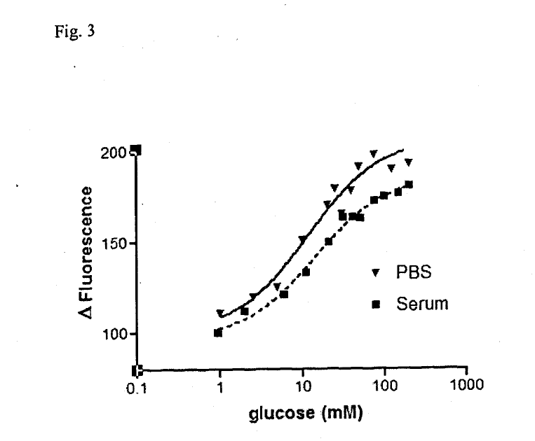

FIG. 3 shows that the fluorescence response to glucose for mutant H152C/A213R/L238S-badan was similar for the assay performed in PBS and when serum from healthy individuals was included in the assay.

### Example 8

### Fluorescence Lifetime Studies

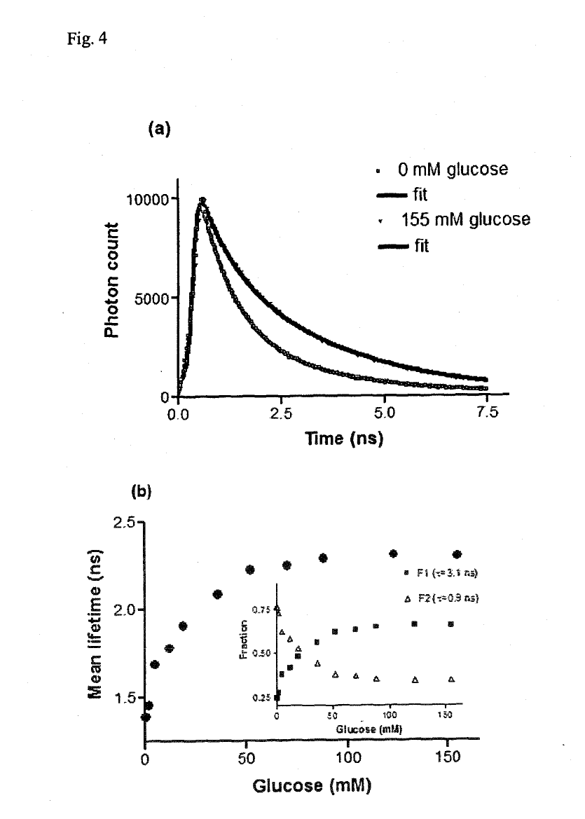

The effect of glucose addition on the fluorescence lifetime of the H152C/A213R/L238S-badan is shown in FIG. 4a and FIG. 4b. A bi-exponential model best fitted the decay curves (FIG. 4a), as determined by the residuals and χ² value. Global analysis of the twelve transients at differing glucose concentrations generated two lifetime states of 3.1 ns and 0.9 ns with a global χ² value of 1.1. Using this model, the fractional contribution of the long lifetime (3.1 ns) increased with glucose and the fractional contribution of the short lifetime state (0.9 ns) decreased (FIG. 4b inset). This can be summarised by the mean fluorescence lifetime, which increased by approximately 1 ns (70%) on addition of saturating glucose (FIG. 4b).

### Example 9

### Immobilisation of GBP

Protocol for Linking GBP (Such as GBP-Badan) to Ni-Polystyrene Beads
1. Resuspend 5 mg polystyrene beads (AM-COOH, 100-200 mesh) in DMSO
2. Add 1.5 mg N-hydroxysuccinimide and 3 mg EDC (ethyl dimethylaminopropyl carbodiimide). Stir at RT for 3 h
3. Centrifuge to remove the DMSO.
4. Wash beads with a phosphate buffer twice, and resuspend in 1 ml 0.1M carbonate buffer pH 8.0
5. Add 3.9 mg 15 mM bis(carboxymethyl)lysine metal chelator (final concentration 15 mM)
6. Stir at RT for 24 h
7. Add 3.9 mg Ni sulphite (final concentration 150 mM)
8. Quench with 10 mM hydroxylamine 1 hour (add 10 ul of 1M stock, 69 mg/ml)
9. Centrifuge and wash twice in PBS
10. Add 200 ul GBP-badan (50 uM in PBS)
11. Incubate o/n 4° C.

NTA agarose beads can be purchased commercially.

GBP-badan may be immobilized in batches according to need.

### REFERENCES

[1]. Klonoff, D. C. Continuous glucose monitoring: Roadmap for 21st century diabetes therapy. Diabetes Care 28 (2005) 1231-9.
[2]. Gross, T. M., Bode, B. W., Einhorn, D., Kayne, D. M., Reed, J. H., White, N. H., Mastrototaro, J. J. Performance evaluation of the MiniMed Continuous Glucose Monitoring System during patient home use. Diabetes Technol. Ther. 2 (2000) 49-56.
[3]. Maran, A., Crepaldi, C., Tiengo, A., Grassi, G., Vitali, E., Pagano, G., Bistoni, S., Calabrese, G., Santeusanio, F., Leonetti, F., Ribaudo, M., Di Mario, U., Annuzzi, G., Genovese, S., Riccardi, G., Previti, M., Cucinotta, D., Giorgino, F., Bellomo, A., Giorgino, R., Poscia, A., Varalli, M. (2002), Continuous subcutaneous glucose monitoring in diabetic patients: a multicentre analysis. Diabetes Care 25 (2002) 347-52.
[4]. Feldman, B., Brasg, R., Schwarz, S., Weinstein, R. A continuous glucose sensor based on Wired Enzyme Technology. Results from a 3-day trial in patients with type 1 diabetes. Diabetes Technol. Therapeut. 5 (2003) 769-79.
[5]. Juvenile Diabetes Research Foundation Continuous Glucose Monitoring Study Group. Continuous glucose monitoring and intensive treatment of type I diabetes. N. Engl. J. Med. 359 (2008) 1464-1476.
[6]. Pickup, J. C., Hussain, F., Evans, N. D. and Sachedina N. In vivo glucose monitoring: the clinical reality and the promise. Biosens. Bioelectron. 20 (2005) 1897-902.
[7]. The Diabetes Research in Children Network (DirecNet) Study Group. Lack of accuracy of continuous glucose sensors in healthy, non-diabetic children: results of the Diabetes Research in Children Network (DirecNet) Accuracy Study. J. Pediatr. 144 (2004) 770-775.
[8]. Pickup, J. C., Hussain, F., Evans, N. D., Rolinski, O. J. and Birch, D. J. Fluorescence based glucose sensors. Biosens. Bioelectron. 20 (2005) 2555-65.
[9]. Moschou, E. A., Sharman, B. V., Deo, S. K., Daunert, S. Fluorescence glucose detection: advances toward the ideal in vivo biosensor. J. Fluoresc. 14 (2004) 535-47.
[10]. Mansouri, S., Schultz, J. S. A miniature optical glucose sensor based on affinity binding. Biotechnology 2 (1984) 885-890.
[11]. Ballerstadt, R., Polak, A., Beuhler, A. and Frye, J. In vitro long-term performance study of a near-infrared fluorescence affinity sensor for glucose monitoring. Biosens. Bioelectron. 19 (2004) 905-914.
[12]. McCartney, L. J., Pickup, J. C., Rolinski, O. J. and Birch, D. J. S. Near infrared fluorescence lifetime assay for serum glucose based on allophycocyanin labelled concanavalin A. Anal. Biochem. 292 (2001) 216-221.
[13]. Chinnayelka, S., McShane M. J. Glucose sensors based on microcapsules containing an orange/red competitive binding resonance energy transfer assay. Diabetes Technol. Therapeut. 8 (2006) 269-78.
[14]. Lakowicz, J. R. (1999), Principles of Fluorescence Spectroscopy, second ed. Plenum Press, New York.
[15]. Birch, D. J. S., Imhof, R. E. Time-domain fluorescence spectroscopy using time-correlated single-photon counting. In: Topics in Fluorescence Spectroscopy, Lakowicz, J. R. (ed), vol 1 (1991), Plenum Press, New York, pp 1-95.
[16]. Marvin, J. S. and Hellinga, H. W. Engineering biosensors by introducing fluorescent allosteric signal transducers-construction of a novel glucose sensor. J. Am. Chem. Soc. 120 (1998) 7-11.
[17]. Tolosa, L., Gryczynski, I., Eichhorn, L. R., Dattelbaum, J. D., Castellano, F. N., Rao, G., Lakowicz, J. R. Glucose sensor for low-cost lifetime-based sensing using a genetically engineered protein. Anal. Biochem. 267 (1999) 114-120.
[18]. Salins, L. L. E., Ware, R. A., Ensor, M., Daunert, S. A novel reagentless sensing system for measuring glucose based on the galactose/glucose binding protein. Anal. Biochem. 294 (2001) 19-26.
[19]. Ye, K., Schultz, J. S. Genetic engineering of an allosterically based glucose indicator protein for continuous glucose monitoring by fluorescence resonance energy transfer. Anal. Chem. 75 (2003) 3451-3459.
[20]. Fehr, M., Lalonde, S., Lager, I., Wolff, M. W., Frommer, W. B. In vivo imaging of the dynamics of glucose uptake in the cytosol of COS-7 cells by fluorescence nanosensors. J. Biol. Chem. 278 (2003) 19127-19133.
[21]. Ge, X., Tolosa, L., Rao, G. Dual-labeled glucose binding protein for ratiometric measurement of glucose. Anal. Chem. 76 (2004) 1403-1410.
[22]. Thomas, K. J., Sherman, D. B., Amiss, T. J., Andaluz, S. A., Pitner, J. B. A long-wavelength fluorescent glucose biosensor based on bioconjugates of galactose/glucose binding protein and nile red derivatives. Diabetes Technol. Therapeut. 8 (2006) 261-68.
[23]. Thomas, K. J., Sherman, D. B., Amiss, T. J., Andaluz, S. A., Pitner, J. B. Synthesis and biosensor performance of a near-IR thiol-reactive fluorophore based on benzothiazolium squaraine. Bioconjug. Chem. 18 (2007) 1841-6.
[24]. Tang, B., Cao, L., Xu, K., Zhuo, L., Ge, J., Li, Q., Yu, L. A new nanobiosensor for glucose with high sensitivity and selectivity in serum based on fluorescence resonance Energy transfer (FRET) between CdTe quantum dots and Au nanoparticles. Chemistry 14 (2008) 3637-44.
[25]. Khan, F., Gnudi, L., Pickup, J. C. Fluorescence-based sensing of glucose using engineered glucose/galactose binding protein: A comparison of fluorescence resonance energy transfer and environmentally sensitive dye labelling strategies. Biochem. Biophys. Res. Commun. 365 (2008) 102-106.
[26]. Saxl, T., Khan, F., Matthews, D. R., Zhi, Z-L., Rolinski, O., Ameer-Beg, S., Pickup, J. C. Fluorescence lifetime spectroscopy and imaging of nano-engineered glucose sensor microcapsules based on glucose/galactose-binding protein. Biosens. Bioelectron. 24 (2009) 3229-34.
[27]. Vyas, N. K., Vyas, M. N., Quiocho, F. A. Sugar and signal-transducer binding sites of the Escherichia coli galactose chemoreceptor protein. Science 242 (1988) 1290-1295.
[28]. Barber, P. R., Ameer-Beg, S. M., Gilbey, J., Carlin, L. M., Keppler, M., Ng, T. C., Vojnovic, B. (2009), Multiphoton time-domain FLIM: practical application to protein-protein interactions using global analysis. J. R. Soc. Interface 6 (2009) S93-S105.
[29]. Dwyer, M. A., Hellinga, H. W. Periplasmic binding proteins: a versatile superfamily for protein engineering. Curr. Opin. Struct. Biol. 14 (2004) 495-504.
[30]. de Lorimier, R. M., Smith, J. J., Dwyer, M. A., Looger, L. L., Sali, K. M., Paavola, C. D., Rizk, S. S., Sadigov, S., Conrad, D. W., Loew, L., Hellinga, H. W. Construction of a fluorescent biosensor family. Protien Sci. 11 (2002) 2655-75.
[31]. Hsieh, H. V., Pfeiffer, Z. A., Amiss, T. J., Sherman, D. B., Pitner, J. B. Direct detection of glucose by surface plasmon resonance with bacterial glucose/galactose-binding protein. Biosens. Bioelectron. 19 (2004) 653-660.
[32]. Sakaguchi-Mikami, A., Taneoka, A., Yamoto, R., Ferri, S., Sode, K. Engineering of ligand specificity of periplasmic binding protein for glucose sensing. Biotechnol. Lett. 30 (2008) 1453-60.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A glucose binding protein comprising amino acid mutations relative to the wild type sequence at the following positions:
(i) H152,
(ii) A213; and
(iii) L238
wherein the mutation at position H152 is H152C.

2. A glucose binding protein according to claim 1 comprising the mutations H152C, A213R and L238S.

3. A glucose binding protein according to claim 1 linked to an environmentally sensitive dye.

4. A glucose binding protein according to claim 3 wherein the environmentally sensitive dye comprises badan (6-bromoacetyl-2-dimethylaminonaphthalene).

5. A microcapsule comprising a glucose binding protein according to claim 1.

6. A fibre optic strand comprising a glucose binding protein according claim 1 or a microcapsule according to claim 5 attached thereto.

7. A nucleic acid encoding a glucose binding protein according to claim 1.

8-14. (canceled)
